(19) United States
(12) Patent Application Publication
(10) Pub. No.: US 2010/0160745 A1
(43) Pub. Date: Jun. 24, 2010

(54) Detection of Food or Drink Consumption In Order to Control Therapy or Provide Diagnostics

(75) Inventors: Matthew Hills, Los Altos, CA (US); Rose Province, San Jose, CA (US); Mir Imran, Los Altos Hills, CA (US); Vince Kapral, Redwood City, CA (US)

Correspondence Address: IntraPace, Inc. & Townsend & Townsend & Crew LLP, Two Embarcadero Center, Eighth Floor, San Francisco, CA, 94111-3834, US

(73) Assignee: IntraPace, Inc., Mountain View, CA (US)

(21) Appl. No.: 12/637,452

(22) Filed: Dec. 14, 2009

Related U.S. Application Data
(60) Provisional application No. 61/122,315, filed on Dec. 12, 2008.

(51) Int. Cl. A61B 5/01; A61B 5/11

(52) U.S. Cl. 600/301; 600/549

(57) ABSTRACT

Methods and systems discriminate between food and drink intake, optionally with a single temperature sensor positioned in a patient's stomach. Ingestion events may be detected and the substance ingested is classified as either food or drink based on several characteristics of the intra-gastric temperature signal from before, during, and after ingestion. Multiple ingestion events making up a meal may be detected and classified such that the entire meal can be classified as food only, drink only, or mixed food and drink. Treatments to a patient may be at least partially based upon the detection and classification of ingestion events. A method of preparing an intake classification algorithm using a training set of temperature data is also provided.

### CROSS-REFERENCES TO RELATED APPLICATIONS

The present application claims the benefit under 35 USC 119(e) of U.S. Provisional Application No. 61/122,315 filed Dec. 12, 2008; the full disclosure of which is incorporated herein by reference in its entirety.

The subject matter of the present application is related to the following applications: U.S. patent application Ser. No. 12/145,430 filed on Jun. 24, 2008 and U.S. patent application Ser. No. 10/950,345 filed on Sep. 23, 2004, both of which are herein incorporated by reference.

### BACKGROUND OF THE INVENTION

### Field of the Invention

Since the mid-seventies, the prevalence of obesity has increased sharply for both adults and children. These increasing rates raise concern because of their implications for Americans' health. Being overweight or obese may increase the risk of many diseases and health conditions, including: hypertension, dyslipidemia (for example, high total cholesterol or high levels of triglycerides), type 2 diabetes, coronary heart disease, stroke, gallbladder disease, osteoarthritis, sleep apnea and respiratory problems, and some cancers (such as endometrial, breast, and colon).

Obesity and its associated health problems have a significant economic impact on the U.S. health care system. Medical costs associated with excess weight and obesity may involve direct and indirect costs. Direct medical costs may include preventive, diagnostic, and treatment services related to obesity. Indirect costs relate to morbidity and mortality costs. Morbidity costs are defined as the value of income lost from decreased productivity, restricted activity, absenteeism, and bed days. Mortality costs are the value of future income lost by premature death.

Many therapies are currently being investigated for treatment of obesity and diseases associated with obesity. To date, the widely used obesity treatments have not been shown to be ideal, particularly for those afflicted with severe obesity. The approaches that have been proposed range from lifestyle coaching to major surgical therapies. Unfortunately, patient compliance can significantly limit the effectiveness of coaching. While surgical approaches can limit the capacity of the patient's gastrointestinal food intake over a set amount of time regardless of compliance, quite sever surgical modifications may have to be imposed to achieve the desired result, potentially limiting the ease with which the patient can ingest when it is appropriate to do so.

More recently, implanted stimulator therapies have been proposed which seek to stimulate the patient in response to actual ingestion so as to limit food intake. Implantable circuitry and electrodes may be capable of transmitting signals to the patient's gastrointestinal tract (or other tissues), and those signals may help to inhibit intake of food. Moreover, the system may include sensors which detect when the patient has ingested food or a beverage, and may even differentiate between the two. Such therapies offer tremendous promise, potentially enforcing a modification of the patient behavior so as to promote a more healthy lifestyle. However, for such a behavior modification to reach its potential, the accuracy with which the system differentiates between different ingested substances should be quite good. In other words, as the correlation between the behavior and the feedback is degraded, the modification of the behavior may suffer tremendously. Moreover, while highly invasive, short term, complex, energy intensive, and/or expensive systems might provide more than the desired differentiation accuracy for such behavior modification, the benefits of such a system might still be limited to very few (if any) actual patients.

Therefore, it would be desirable to provide devices, systems and methods that can effectively promote behavior modification of patients suffering from obesity and other eating disorders. It would also be desirable to provide improved detection and classification of the ingestion of food or drink by a patient. Ideally, such a system would enhance the accuracy with which the system can differentiate between different types of ingested materials without having to resort to more complex sensors systems so as to avoid at least some of the short-comings of known methods and devices.

### BRIEF SUMMARY OF THE INVENTION

The present invention relates to the detection and classification of the intake of food or drink by a patient. Although embodiments make specific reference to such identification and classification within an obesity treatment setting, the system and methods described herein may be applicable to any function in which detection and classification of ingestion is desired. Embodiments of the present invention provide a method and system for discriminating, for example, between food and drink intake with a single temperature sensor positioned in the stomach of the patient. Using temperature measurements obtained from the temperature sensor, it is possible to detect when an ingestion event has occurred and to classify the substance ingested as either food or drink based on several characteristics of the intra-gastric temperature signal from before, during, and after ingestion. In many embodiments, multiple ingestion events making up a meal are detected and classified such that the entire meal can be classified as food only, drink only, or mixed food and drink. In some embodiments, methods and systems are provided for treating a patient based upon the detection and classification of ingestion events. In other embodiments, a method of preparing a classification algorithm using a training set of temperature data is provided. Still further embodiments may enhance the benefits from additional and/or other types of sensors so as to differentiate between a variety of classes of ingestion.

The ability to determine when a patient consumes a meal and to identify the type of meal consumed has advantages from both therapy control and diagnostic perspectives. In terms of therapy control, the identification of a drink only meal may be used to trigger early termination of the therapy or removal of a therapy refractory period. The purpose of a refractory period is to ensure that further ingestion events are not detected during recovery of the temperature to a baseline value. The detection of the end of a meal could trigger a shortening or termination of a refractory period. In terms of diagnostics, for example, it may be desired to report a parameter that is correlated to a patient's total calorie intake, such as the total meal duration for a 24-hour period, which can be considered a good qualitative estimate of calorie intake even without the advantage of further sensor data to determine meal composition.

In a first aspect, embodiments of the present invention provide a method of classifying ingestion by a patient. The method includes obtaining a plurality of stomach temperature sample values associated with a plurality of time intervals. The temperature values may be stored in a buffer and it is determined whether an ingestion event has occurred using the stored temperature values in order to determine whether classification is to be performed. The ingestion event is then classified as eating or drinking using the stored temperature values.

In some embodiments, the buffer stores a predetermined number of temperature values that define a sampling window. The step of determining whether an ingestion event has occurred may include segmenting the sampling window into first, second and third time periods, determining first and second averages of the temperature values for the first and second time periods, comparing the first and second averages, and determining whether the difference between the first and second averages exceeds a predetermined threshold.

In some embodiments, the step of classifying the ingestion event includes analyzing features of the temperature values in the sampling window. The step of classifying the ingestion event may also include using a linear separator to classify the ingestion event, using a non-linear separator to classify the ingestion event, and/or weighting each of the analyzed features with an associated weight. The analyzed features may include a mean of the temperature values, a sum of the absolute values of sample-to-sample temperature differences, a variance of the temperature values, an area under a back half of a waveform defined by the temperature values in the sampling window, an energy in a front half of the waveform, an energy in the back half of the waveform, and a maximum temperature difference of the temperature values. Most often, more than two of the features will be analyzed in classifying the ingestion event, preferably more than three features, and more preferably more than four. In a most preferred embodiment, more than five of the features will be analyzed.

In some embodiments, the steps of determining whether an ingestion event has occurred and of classifying the ingestion event are performed using a single set of temperature values that define a single sample window. In other embodiments, the step of determining whether an ingestion event has occurred uses a first set of temperature values that define a first sampling window and the step of classifying the ingestion event uses a second set of temperature values that define a second sampling window.

In some embodiments, the method may also include obtaining additional temperature values and updating the buffer with the additional temperature values when it is determined that the temperature values are not to be classified or that an ingestion event has not occurred.

In a second aspect, embodiments of the present invention provide a method of classifying a meal ingested by a patient that includes detecting a first ingestion event using at least one sensor disposed within a patient. A meal timer is started in response to the event detection, the first ingestion event is classified and the classification is recorded. Subsequent ingestion events are detected and classified and the classifications are recorded until a predetermined period of time has passed without an event detection. A meal duration is recorded in response to the time without an event detection and the meal is classified in response to the signals from the at least one sensor.

In some embodiments, classifying the ingestion event includes classifying the event as eating or drinking.

In some embodiments, classifying the meal includes classifying the meal as food only, drink only, or mixed food and drink.

In some embodiments, the method also includes determining an activity level of the patient, and setting the meal classification to drink only in response to the activity level of the patient indicating that the patient is exercising.

In some embodiments, the meal classification is set to drink only where a signal from the sensor returns toward a pre-ingestion level in less than a predetermined period or the meal duration is shorter than a predetermined period.

In a third aspect, embodiments of the present invention provide a method of classifying a meal ingested by a patient that includes obtaining a baseline stomach temperature of the patient, waiting for an ingestion event, detecting a first ingestion event, classifying the first ingestion event as food or drink and storing the classification. Where the classification of the first ingestion event is drink, a maximum deviation of the stomach temperature from the baseline temperature and a maximum recovery slope of the stomach temperature are determined and stored, an end of the meal, a meal duration and whether the recovery slope exceeds a predetermined threshold are determined, and the meal is classified as drink only or mixed food and drink. Where the classification of the first ingestion event is food, whether a subsequent ingestion event is classified as drink and an end of the meal are determined, and the meal is classified as food only or mixed food and drink.

In some embodiments, determining the end of the meal includes determining that the stomach temperature is within a predetermined range of the baseline temperature or that no event detection has occurred within a predetermined period of time.

In some embodiments, the method also includes storing a timestamp for a start of the meal.

In some embodiments, determining the end of the meal includes storing a timestamp of the end of the meal.

In some embodiments, the meal classification is set to drink only where the classification of the first ingestion event is drink and the meal duration is less than a first predetermined duration.

In some embodiments, the meal classification is set to drink only where the classification of the first ingestion event is drink, the meal duration is less than a second predetermined duration and the recovery slope exceeds a predetermined threshold.

In some embodiments, the method also includes obtaining a stomach temperature value when the first ingestion event is detected, comparing the temperature value to a core body temperature, and determining whether to accept the first ingestion event or to return to waiting for an ingestion event.

In some embodiments, obtaining the baseline stomach temperature of the patient includes storing a timestamp of the most recent event detection, determining if a predetermined period of time has passed since the most recent event detection, determining an activity level of the patient, and when the predetermined period of time has passed and the activity level of the patient is low, recording stomach temperature values over a period of time and averaging the temperature values to obtain a baseline stomach temperature.

In a fourth aspect, embodiments of the present invention provide a method of treatment of a patient that includes detecting a first ingestion event and classifying the ingestion event as food or drink. Where the ingestion event is classified as drink, a first therapy is provided to the patient, and where the ingestion event is classified as food, a second therapy is provided to the patient.

In some embodiments, the method also includes providing a first refractory period to the patient after the first therapy and providing a second refractory period to the patient after the second therapy.

In some embodiments, the method also includes ending the first or second therapies or the first or second refractory periods when an end of a meal is detected.

In some embodiments, the method also includes detecting subsequent ingestion events, where the first and subsequent ingestion events define a meal, classifying the meal, and, where the first ingestion event is classified as drink and the meal is classified as mixed food and drink, ending the first therapy to the patient and providing the second therapy to the patient.

In a fifth aspect, embodiments of the present invention provide a system for classifying ingestion by a patient that includes a temperature sensor adapted to be placed in the stomach of the patient, a storage medium connected to the sensor for storing temperature values, and a processor connected to the storage medium that is configured to analyze the temperature values, where the processor includes a module for determining whether the temperature values are to be classified, a module for determining whether an ingestion event has occurred and a module for classifying the ingestion event as eating or drinking.

In some embodiments, the processor includes a tangible medium embodying instructions for analyzing the temperature values, determining whether the temperature values are to be classified, determining whether an ingestion event has occurred and classifying the ingestion event.

In a sixth aspect, embodiments of the present invention provide a system for classifying a meal ingested by a patient that includes a temperature sensor adapted to be placed in the stomach of the patient, a meal timer, an activity sensor, a storage medium connected to the temperature sensor, the meal timer and the activity sensor, and a processor connected to the storage medium that is configured to analyze temperature values, timestamps and activity level data stored in the storage medium to classify the meal.

In a seventh aspect, embodiments of the present invention provide a system for classifying a meal ingested by a patient that includes a temperature sensor adapted to be positioned in the stomach of the patient, a storage medium connected to the temperature sensor, and a processor connected to the storage medium that is configured to analyze temperature values stored in the storage medium to classify the meal, where the processor includes a first module for determining a baseline stomach temperature of the patient, a second module for classifying a first ingestion event as food or drink based on the temperature values, and a third module for classifying the meal, where when the classification of the first ingestion event is drink, the third module determines and stores a maximum deviation of the stomach temperature from the baseline temperature, determines and stores a maximum recovery slope of the stomach temperature, determines an end of the meal and a meal duration, determines whether the recovery slope exceeds a predetermined threshold, and classifies the meal as drink only or mixed food and drink, and when the classification of the first ingestion event is food, the third module determines if a subsequent ingestion event is classified as drink, determines an end of the meal and classifies the meal as food only or mixed food and drink.

In a eighth aspect, embodiments of the present invention provide a system for treatment of a patient that includes a temperature sensor adapted to be positioned in the stomach of the patient, a storage medium connected to the temperature sensor, a therapeutic device adapted to provide at least one therapy to the patient, and a processor connected to the storage medium and the therapeutic device that is configured to analyze temperature values stored in the storage medium to classify the meal and to control the therapeutic device based on the classification.

In a ninth aspect, embodiments of the present invention provide a system for classifying ingestion by a patient that includes means for obtaining a plurality of stomach temperature sample values, means for storing the temperature values, and means for analyzing the stored temperature values, where the means for analyzing includes means for determining whether the stored temperature values are to be classified, means for determining whether an ingestion event has occurred using the stored temperature values, and means for classifying the ingestion event as eating or drinking using the stored temperature values.

In a tenth aspect, embodiments of the present invention provide a method of preparing a classification system for patient ingestion that includes providing training sets of temperature data to a classification algorithm, where the training sets correspond to known activities, determining a set of features of the temperature data, determining a set of weights corresponding to the set of features using the temperature data and the corresponding known activities, and deriving a classification algorithm from the set of features and the set of weights.

In some embodiments, the method also includes determining an event parameter threshold and a bias value and incorporating the event parameter threshold and the bias value into the classification algorithm. Determining the bias value and determining the set of weights may include using a support vector machine. Determining the bias value and determining the set of weights may also include optimizing the bias value and the set of weights to provide a maximum separation between the waveforms corresponding to eating and drinking.

In some embodiments, the known activities include no consumption, eating, and drinking, where eating and drinking are defined as screening functions. The training sets may comprise 32-sample data sets. Determining the event threshold parameter may include calculating the mean temperatures for first and second sample subsets of the data sets corresponding to each of the screening functions, determining the absolute difference in the mean temperatures, determining the standard deviation of the screening function values from the no consumption values, and determining the event threshold.

In some embodiments, the set of features to which the weights correspond include a mean of the temperature values, a sum of the absolute values of sample-to-sample temperature differences, a variance of the temperature values, an area under a back half of a waveform defined by the temperature values in the sampling window, an energy in a front half of the waveform, an energy in the back half of the waveform, and a maximum temperature difference of the temperature values. Most often, the set of features will include more than two of the above features, preferably more than three features, and more preferably more than four. In a most preferred embodiment, the set will include more than five of the features.

In an eleventh aspect, embodiments of the present invention provide a method of providing therapy to a patient that includes providing a therapy device with a schedule of allowed and disallowed periods for the patient. For each allowed period, a first therapy is applied to the patient at the start of the period. Any ingestion events during the period are detected using at least one temperature sensor that is disposed within the patient and classified as food or drink. Where an ingestion event is classified as drink, the first therapy is stopped and a second therapy is provided to the patient. Where an ingestion event is classified as food, the first therapy is stopped and a third therapy is provided to the patient. For each of the disallowed periods, the patient is monitored to detect any ingestion events, and any events are classified as food or drink. Where an ingestion event is classified as drink, the second therapy is provided to the patient and where an ingestion event is classified as food, the third therapy is provided.

### BRIEF DESCRIPTION OF THE DRAWINGS

FIG. 1 illustrates an embodiment of a stimulation system of the present invention.

FIG. 2 illustrates another embodiment of a stimulation system of the present invention.

FIGS. 3A and 3B show an equivalent circuit and temperature graph for a heat model of ingestion.

FIGS. 4A and 4B show temperature deviations for different types of meal events.

FIG. 5 illustrates an algorithm of event classification according to an embodiment of the present invention.

FIG. 6 shows a sample buffer window for event detection according to an embodiment of the present invention.

FIG. 7 illustrates an algorithm of meal classification according to an embodiment of the present invention.

FIG. 8 illustrates an algorithm of meal classification according to another embodiment of the present invention.

FIG. 9 illustrates an algorithm for updating a baseline body temperature according to an embodiment of the present invention.

FIG. 10 illustrates a therapy control method according to an embodiment of the present invention.

### DETAILED DESCRIPTION OF THE INVENTION

The present invention relates to the detection and classification of the intake of food or drink by a patient. In most instances, patients suffering from obesity have diminished ability to self-manage their daily food intake. Patients often overeat, snack between meals and generally make poor food choices. In order to effectively apply therapies that are being used to treat obesity and related diseases, many of which are optimally delivered at the time of ingestion of food or drink, it is advantageous to be able to detect an ingestion event and to accurately classify the type of ingestion event.

Embodiments of the present invention use temperature measurements obtained from a temperature sensor positioned in the stomach of the patient to detect when an ingestion event has occurred and to classify the substance ingested as either food or drink. In many embodiments, multiple ingestion events making up a meal are detected and classified such that the entire meal can be classified as food only, drink only, or mixed food and drink. In some embodiments, methods and systems are provided for treating a patient based upon the detection and classification of ingestion events. In other embodiments, a method of preparing a classification algorithm using a training set of temperature data is provided. Alternative embodiments may augment (or in some cases, even replace) the temperature data with information from other sensors. For example, event detection and/or classification might instead be based at least in part on spectroscopic data, signals generated by electromechanical sensors coupled to the stomach or esophagus, electrical impedance data, and/or the like. Additional data from these or other sources may be combined with the techniques described herein to enhance the ability of the system to promote healthy behavior by discriminating between additional classes of ingestion, such as between low fat and high fat materials, between low carbohydrate and high carbohydrate materials, between low protein and high protein materials, and the like. Nonetheless, the information available from a simple, reliable, low-energy consumption temperature sensor particularly such a sensor disposed within the stomach of a patient may provide a significant amount of information regarding the classes of material ingested by the patient.

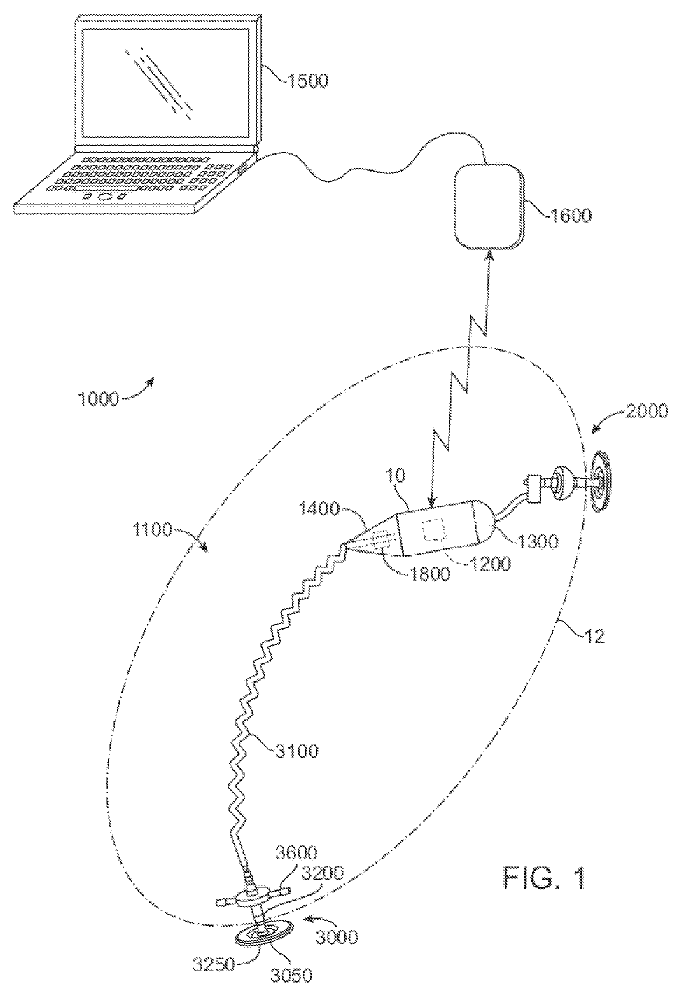

An example system 1000 suitable for implementation of embodiments of the present invention is illustrated in FIG. 1. In the embodiment shown, the system 1000 comprises a stimulator 1100 which is implantable within an organ such as a stomach 12, small intestine or colon. The stimulator 1100 comprises implantable electronic circuitry 1200 contained within an implantable pulse generator (IPG) 10 which typically has a protective housing 1300. The housing 1300 is constructed of a corrosion resistant material, such as a material able to withstand implantation within a gastric environment. An IPG anchor 2000 is coupled to the IPG 10 and is configured to anchor the IPG 10 to a wall of the stomach. The stimulator 1100 also includes an electrode lead anchor 3000 comprising a first electrode 3200 and a return electrode 3250. The electrodes 3200, 3250 are coupled to the electronic circuitry 1200 through a flexible lead portion 3100 to a connector 1800 within header 1400 of housing 1300. The electrode lead anchor 3000 is configured to anchor the electrode 3200 so that it is in electrical contact with, or in proximity to the stomach wall 12. The electronic circuitry 1200 is configured to provide an electrically stimulating signal to a stomach wall via the electrodes 3200, 3250. While the electrodes 3200, 3250 are shown in particular configurations and locations, numerous electrode configurations and positions are contemplated. An external computer or programmer 1500 may be used to program various stimulation parameters or other instructions into a memory device included with the electronic circuitry 1200. The external programmer 1500 may be coupled to a telemetry device 1600 that communicates with the electronic circuitry via radio frequency signals.

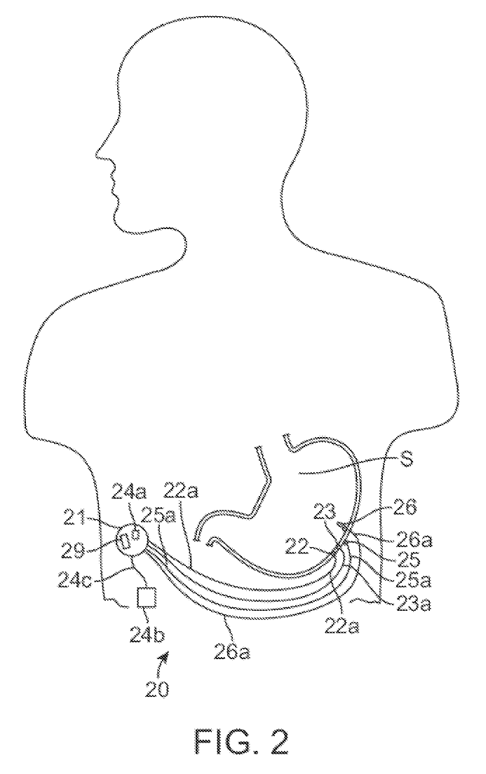

FIG. 2 illustrates another example of a stimulation system. This embodiment includes a stimulator 20 having an implantable pulse generator (IPG) 21 implanted subcutaneously within a patient. The stimulator further comprises leads 22a, 23a extending from the IPG 21 through the abdomen and to the stomach S where electrodes 22, 23 are implanted into the stomach muscle layer from the outside of the stomach S. The IPG 21 further comprises a sensor 24a located on the IPG 21 and/or a sensor 24b may be separate from the IPG and located elsewhere in the patient and coupled to the electronic circuitry 29 in the IPG by lead 24c. The stimulator also includes sensors 25, 26, that are implanted on or in the stomach S, respectively, with leads 25a, 26a extending from the sensors 25, 26 to the IPG 21. Sensor 26 is exposed to the inside of the stomach S while sensor 25 is attached to the outside of the stomach. Leads 22a, 23a, 24c, 25a, 26a are electrically coupled to the electronic circuitry 29 located in the IPG 21.

In the present invention, the gastric stimulators include or are used with at least one temperature sensor for sensing temperature information. The sensors may be located on or extend from the IPG and/or the sensors may be located on or extend from a lead or other device. Alternatively or additionally, a sensor may be located separately on the stomach wall and/or a sensor may be otherwise positioned elsewhere within, coupled to or in communication with the patient. In some embodiments, data obtained from the sensor may be pre-processed to remove noise or unwanted artifacts before it is analyzed.

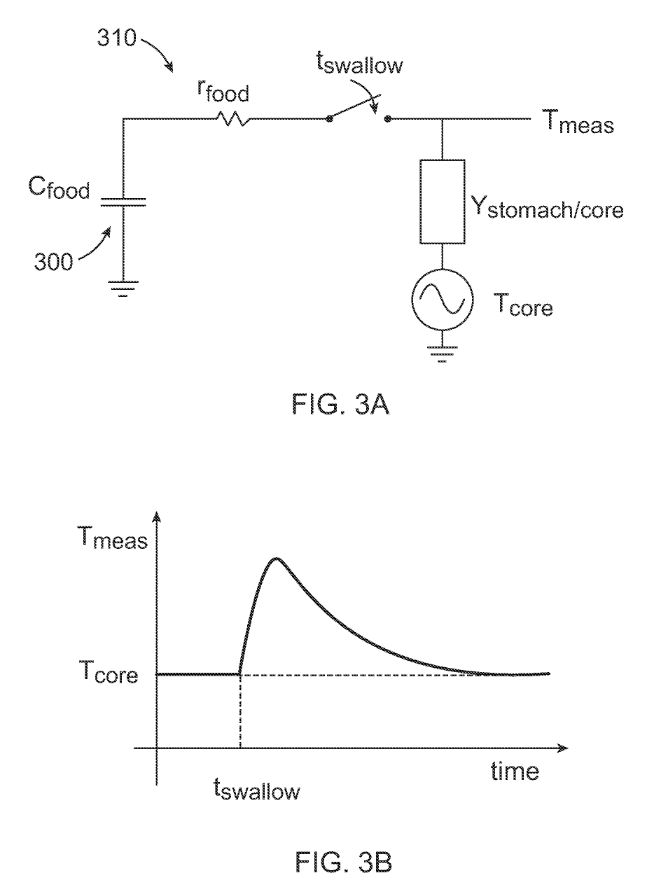

The potential for using temperature measurements to classify ingestion events can be understood from a simple heat model, illustrated by the equivalent circuit shown in FIG. 3A. When hot food or liquid (represented by C_{food} 300) is swallowed, it will be introduced to the stomach with excess heat (represented by the charge on: Q_{food}=C_{food} T_{food}). The stomach will rapidly rise in temperature (as shown in FIG. 3B), then gradually equilibrate to the core body temperature. The resistance r_{food} 310 models the effective heat transfer from the food to the stomach. This includes both actual heat resistances as well as phenomena such as churning of the stomach contents. It is expected that r_{food} 310 will be significantly lower for liquids, resulting in much faster transients.

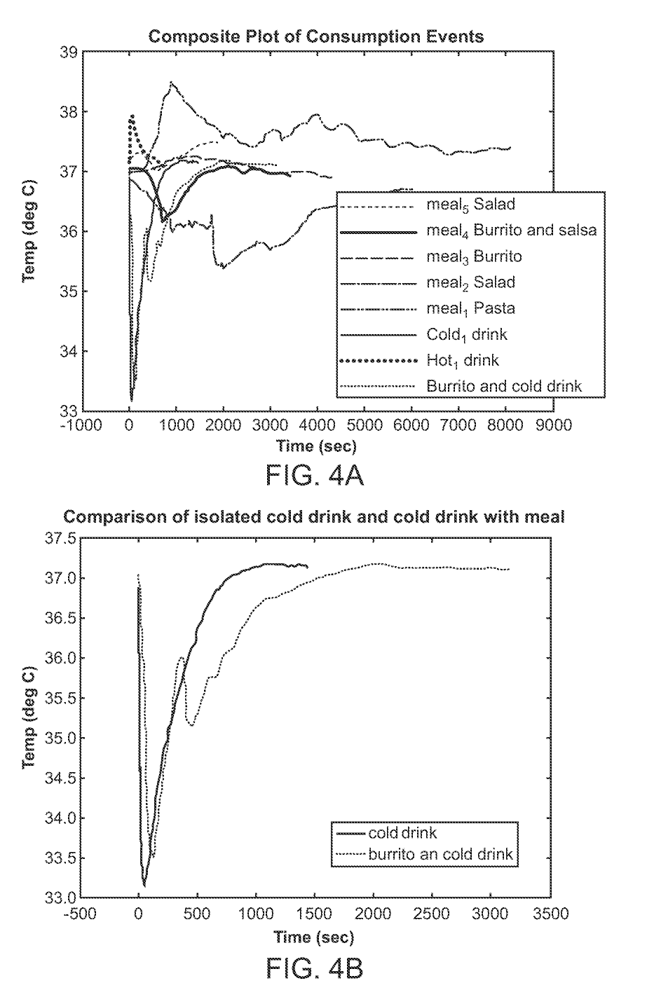

The first-order model suggests that the equilibration is exponential, with a characteristic time constant that will depend on C_{food} 300 and r_{food} 310, but not the temperature of the food. The peak temperature will depend on all three of these. Consequently, the consumption of liquid will generally exhibit sharper peaks that decay more quickly than the consumption of food. Similarly, measures of parameters like signal energy would be higher with fluid consumption and lower with food. The core temperature does not change as rapidly as the stomach temperature, thus a shift in stomach temperature over a short time frame can be understood as having been caused by consuming something hot or cold, which provides the basis for identifying an ingestion event. Further, as shown in FIGS. 4A and 4B, the recovery time for the stomach to return to a baseline temperature can be used to differentiate between meals with food and meals with drink only. Another factor impacting the recovery time is the difference in the speeds at which liquids and solid foods travel through the stomach. In many embodiments, the sensor is generally closer to the proximal part of the stomach, allowing either food or drink entering the stomach to be detected quickly. But after that initial detection, liquids will travel through the regions of the stomach at a faster speed and will be digested at a faster rate in comparison with solid foods, thus the stomach temperature will equilibrate faster after ingestion of liquids, shortening the observed recovery time.

In the embodiment of FIG. 1, circuitry 1200, telemetry device 1600, and external programmer 1500 are included in a data processing system of system 1000. Similarly, in the embodiment of FIG. 2, circuitry 29 may comprise a stand alone data processing system or may be configured to interface with one or more additional electronic components external of (and/or implanted at different locations within) the patient. Generally, the data processing systems included in embodiments of the invention may include at least one processor, which will typically include circuitry implanted in the patient, circuitry external of the patient, or both. When external processor circuitry is included in the data processing system, it may include one or more proprietary processor boards, and/or may make use of a general purpose desktop computer, notebook computer, handheld computer, or the like. The external processor may communicate with a number of peripheral devices (and/or other processors) via a bus subsystem, and these peripheral devices may include a data and/or programming storage subsystem or memory. The peripheral devices may also include one or more user interface input devices, user interface output devices, and a network interface subsystem to provide an interface with other processing systems and networks such as the Internet, an intranet, an Ethernet™, and/or the like. Implanted circuitry of the processor system may have some or all of the constituent components described above for external circuitry, with peripheral devices that provide user input, user output, and networking generally employing wireless communication capabilities, although hard-wired embodiments or other transcutaneous telemetry techniques could also be employed.

An external or implanted memory of the processor system will often be used to store, in a tangible storage media, machine readable instructions or programming in the form of a computer executable code embodying one or more of the methods described herein. The memory may also similarly store data for implementing one or more of these methods. The memory may, for example, include a random access memory (RAM) for storage of instructions and data during program execution, and/or a read only memory (ROM) in which fixed instructions are stored. Persistent (non-volatile) storage may be provided, and/or the memory may include a hard disk drive, a compact digital read only memory (CD-ROM) drive, an optical drive, DVD, CD-R, CD-RW, solid-state removable memory, and/or other fixed or removable media cartridges or disks. Some or all of the stored programming code may be altered after implantation and/or initial use of the device to alter functionality of the system.

The functions and methods described herein may be implemented with a wide variety of hardware, software, firmware, and/or the like. In many embodiments, the various functions will be implemented by modules, with each module comprising data processing hardware and/or software configured to perform the associated function. The modules may all be integrated together so that a single processor board runs a single integrated code, but will often be separated (such as between an implanted processor board resident within the patient and an external processor board of a laptop or the like wirelessly coupled to the implanted board) so that, for example, more than one processor board or chip or a series subroutines or codes are used. Similarly, a single functional module may be separated into separate subroutines or be run in part on separate processor chip that is integrated with another module. Hence, a wide variety of centralized or distributed data processing architectures and/or program code architectures may be employed within different embodiments.

The electronic circuitry comprises and/or is included within a controller or processor for controlling the operations of the device, including sensing, stimulating, signal transmission, charging and/or using energy from a battery device for powering the various components of the circuit, and the like. As such, the processor and battery device are coupled to each of the major components of the implanted circuit. In some embodiments, the electronic circuitry includes an internal clock. The internal clock may also include a real time clock component. The internal clock and/or real time clock may be used to control stimulation, e.g., by stimulating or allowing stimulation at a particular time of the day. The real time clock component may also provide a date/time stamp for detected events that are stored as information in a memory device. Optionally, the memory may be preserved by saving information corresponding to an event of interest which is saved along with the time/date when the event occurred.

The memory device is configured to store a plurality of code modules for execution by the processor. The code modules provide a variety of determinations based on sensor information and various other inputs, such as information from the internal clock, which may be used to actuate a stimulation driver. A stimulation driver may be coupled to stimulating electrodes that are used to provide electrical stimulation therapy to a patient.

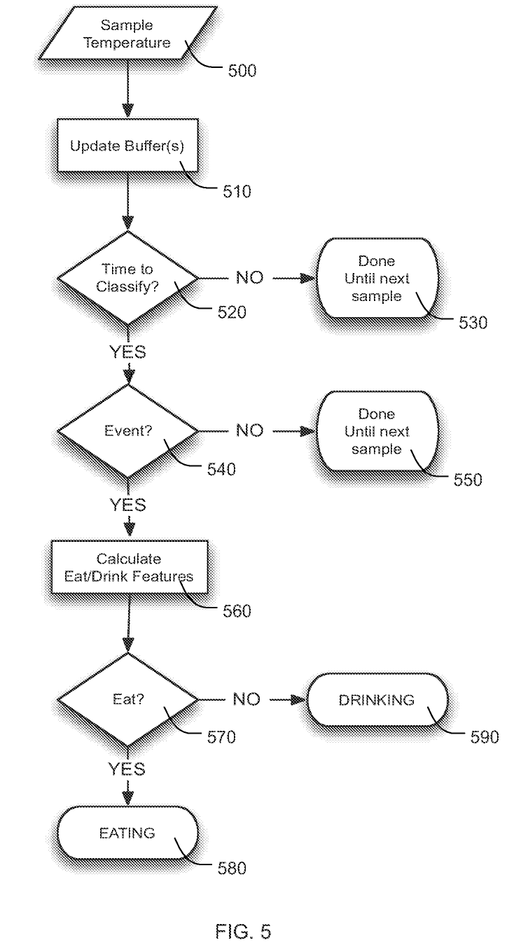

FIG. 5 illustrates a method of classifying an ingestion event according to an embodiment of the present invention. With a temperature sensor positioned in the stomach of the patient, the stomach temperature is sampled at regular time intervals (step 500) and maintained in a buffer that holds a plurality of the most recent temperature samples. In a preferred embodiment, the temperature is sampled every 6 seconds and maintained in a buffer that holds the most recent 32 temperature samples. This data is analyzed to determine if an ingestion event has occurred, and if one has, then to classify it as an eating or drinking event.

The temperature buffer needs to be valid (i.e. it needs to have all 32 data positions filled) at any time the classification algorithm is run. At system start-up, the buffer is filled with the first temperature measured, then the buffer positions are updated as additional data is acquired from subsequent measurements. During stimulation, or other periods where the classification determination may not be made, it is recommended that the temperature data still be recorded and maintained in the buffer.

After the buffer is updated in step 510, it is determined whether it is time to classify the event (step 520). This step is used to lock out the classification when new stimulation triggers are not wanted, such as after an event has been detected and therapy initiated. This is particularly important because the stomach contents may not return to baseline for an extended period, during which time it can be difficult to classify the waveform accurately. If classification will not occur, the algorithm is finished (step 530) until the next sample is taken. After determining that it is time to classify, step 540 determines whether an event has occurred.

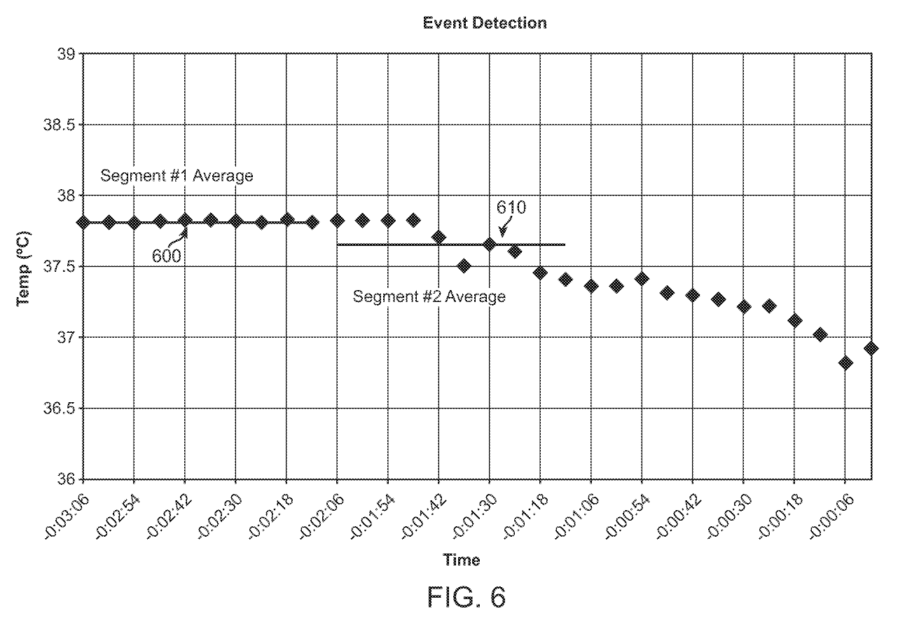

For this event detection, a thresholding algorithm is used. In some embodiments, the temperature buffer is segmented into approximate thirds and the averages of the first two segments are calculated and compared. If the difference exceeds a threshold, a consumption event is declared to have occurred; if not, the algorithm is again finished until the next sample (step 550). Other thresholding algorithms may also be used. For example, the absolute maximums and absolute minimums of the first two segments of the buffer may be determined and the difference compared to a threshold or the maximum slope of the first two segments may be compared to a threshold. In FIG. 6, a sample buffer is shown with the segment averages 600 and 610 indicated. Only the first 20 data points stored in the buffer are used in event detection, then once an event is detected, the data from the entire buffer is used for classification. This approach allows additional data points (12 points, as shown here) to be collected and stored in the buffer after a temperature change has occurred before the system attempts to classify the event, which increases the amount of information available for classification, and correspondingly, increases the accuracy.

As described for these embodiments, the sample window in which the event was detected is also used for the event classification; however, it is not necessary that the same window be used. In other embodiments, an event may be detected in a first sample window, and then classification may be performed on a second sample window. The second sample window may follow the first sample window or overlap with a portion of the first sample window. Consider the example from FIG. 6, in which the buffer holds a sample window of 32 data points. The event detection may proceed as described above, using the first 20 of the 32 data points, but instead of then using those 32 data points from that window for the classification, the system may wait until a new set of 32 data points has been stored in the buffer (e.g., the next 32 data points immediately following the last point shown in FIG. 6). After these data points have been stored, the system defines that new set of points as the sample window for classifying the event. Alternatively, instead of waiting for a completely new set of 32 data points, the system may define a sample window for the classification that overlaps with the sample window from the event detection, such that part of the window includes newly stored data points. For example, the sample window used for classification could include the last 16 points of the event detection sample window, plus the first 16 newly stored data points that follow.

Returning to FIG. 5, the detected event is then classified as eating (food) or drinking (drink). To distinguish eating waveforms from drinking waveforms, features may be calculated from the buffer (step 560) and a linear separator may be used to make the classification in step 570. In some embodiments, seven features are calculated as follows:

a mean of the temperature values,

$${f}_{1} = ( T ) = \overline{T} = \frac{1}{32} \sum {T}_{i} ;$$

a sum of the absolute values of sample-to-sample temperature differences,

$${f}_{2} ( T ) = {\sum }_{i = 1}^{N - 1} abs ( {T}_{i + 1} - {T}_{i} ) ;$$

a variance of the temperature values,

$${f}_{3} ( T ) = \frac{1}{N} {\sum }_{i = 1}^{N} {( {T}_{i} - \overline{T} )}^{2} ;$$

an area under a back half of a waveform defined by the temperature values in the sampling window, f₄(T)=Σ_{i=(N/2)+1}^{N} abs(Tᵢ− T̅);

an energy in a front half of the waveform, f₅(T)=Σᵢ₌₁^{N/2}(Tᵢ₊₁−Tᵢ)²;

an energy in the back half of the waveform, f₆(T)=Σ_{i=(N/2)}^{N}(Tᵢ−Tᵢ₋₁)²; and

a maximum temperature difference of the temperature values, f₇(T)=max(T)−min(T).

Other features describing the characteristics of the temperature signal, such as a median temperature value or an average slope, may also be used for classification. In some embodiments, a weight of zero is given to the mean of the temperature values in order to remove dependence upon the absolute temperature. With this approach, a change in the core body temperature will not affect the treatment of the patient. Other features may be included to account for such core temperature changes, such as features incorporating absolute temperature values or the direction of change of temperature values (i.e., whether the temperature increased or decreased). In some embodiments, a non-linear separator, such as a separator based upon multiple functions, may be used in place of the linear separator. An advantage of the linear separator is the ease of implementation with high computational efficiency; however, other separators might be advantageous where computational efficiency is less critical.

The classification is then determined by multiplying each of the features by an associated weight and adding a bias term: C(T)=−b+Σᵢ₋₁⁷wᵢfᵢ(T), where the classification is food for C(T)>0 (step 280) and drink for C(T)≦0 (step 290). The weights used here are calculated from a set of labeled training data. The training procedure is described in further detail below. This classification method can be used to detect and classify each ingestion event.

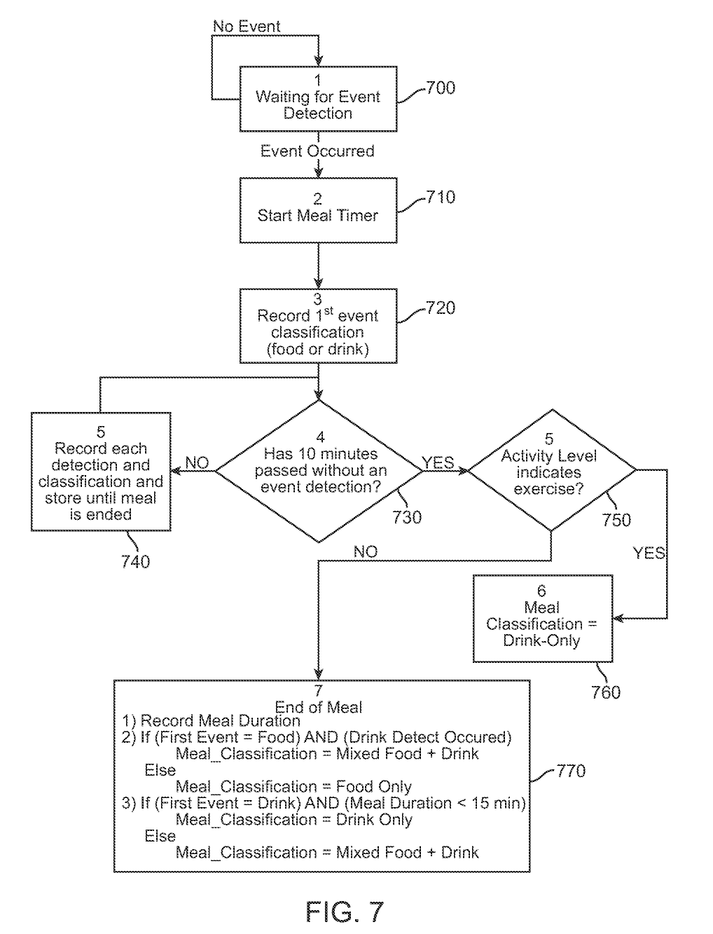

FIG. 7 illustrates an algorithm for defining a meal according to one embodiment of the present invention. Using the temperature-based event classification algorithm described above, a timer and temporary storage of the event classifications for a particular meal are added, along with stored diagnostic parameters for each meal. Initially, the controller waits for an event detection (step 700), where the event detection is defined by the event threshold discussed above. When an event detection occurs, a start of meal timestamp is saved or a meal timer is started (step 710). In addition, the first event classification will be stored for the meal (step 720), and all subsequent event classifications will be stored until the meal is ended (step 740), which is defined by a time, x, without an event detection (step 730). In some embodiments, x is set in the range of 6 to 10 minutes. When the meal has ended, the meal duration is determined based upon the time between the first event detection and the last event detection within the meal.

Before the meal is classified, the level of activity of the patient may be determined (step 750) with an accelerometer, a heart rate monitor, or by communication with an external device that can detect activity. If there is a level of activity that indicates exercise, then the meal will be forced to a drink only classification (step 760), as it is unlikely that a subject is eating food of any significant calorie content while exercising. Otherwise, if exercise is not occurring, classification of the meal begins (step 770) with the classification of the first ingestion event. Where the first event detected was classified as food, there are two meal classification options: food only and mixed food and drink. If no subsequent events were classified as drink, then the meal is classified as food only. If at least one subsequent event was classified as drink, then the meal is classified as mixed food and drink. Similarly, where the first ingestion event was classified as drink, the meal classification options are drink only and mixed food and drink. If the meal duration was less than a predetermined period, for example, 15 minutes, then the meal classification is drink only. Otherwise, if the meal duration was longer, then the meal classification is set to mixed food and drink.

Accordingly, several parameters are stored for each meal, including the meal start time, the meal duration, and the meal classification. The meal start time is the timestamp that corresponds to the start of each meal. The duration of the meal is calculated as described above and, in one embodiment, has an 18-second resolution based upon the frequency of the event classification update. For each meal, one of a food only, a drink only, or a mixed food and drink classification will be stored. In addition to these stored parameters, several diagnostics may be calculated from the stored data on a daily, weekly or monthly basis, including the number of meals per day, the total daily consumption, the number of meals during disallowed periods per day, and the number of possibly undetected meals per day.

These diagnostics provide to a patient and his physician information regarding how his consumption changes on a daily or weekly basis, or if the patient is showing overall improvement in reducing consumption over time. The number of meals that occurred within a 24-hour period and the timestamp for each meal can provide information regarding the patient's daily habits, particularly with respect to the times of day when the patient is most vulnerable to overeating. The total daily consumption is calculated based upon the sum of the meal durations of all the meals detected in a 24-hour period. This calculation provides a measure of the total time spent eating, which can be considered proportional to calorie intake. In some cases, the calculation may involve weighting a drink only meal significantly less than a food only or mixed food and drink meal (e.g., a drink only meal might be weighted by one-third of the weight of the other meals), because it is unlikely that a drink holds equivalent calories to food, and patients trying to lose weight often drink only water under instructions from their physicians. To provide a meaningful consumption index the total daily consumption may be presented as a percentage of the recommended total meal duration.

In addition, when this meal classification algorithm is combined with a therapeutic device, as is discussed in more detail below, the therapeutic device may allow for programming of periods of time in a 24-hour clock when the patient is expected to eat, and periods when it is not recommended that the patient eats. In such cases, it may be of interest to include in the diagnostics the number of meals that occurred during the times when the patient was not supposed to eat (the number of meals during disallowed periods per day), and meals where the patient was expected to eat but no event detection occurred (the number of possibly undetected meals per day).

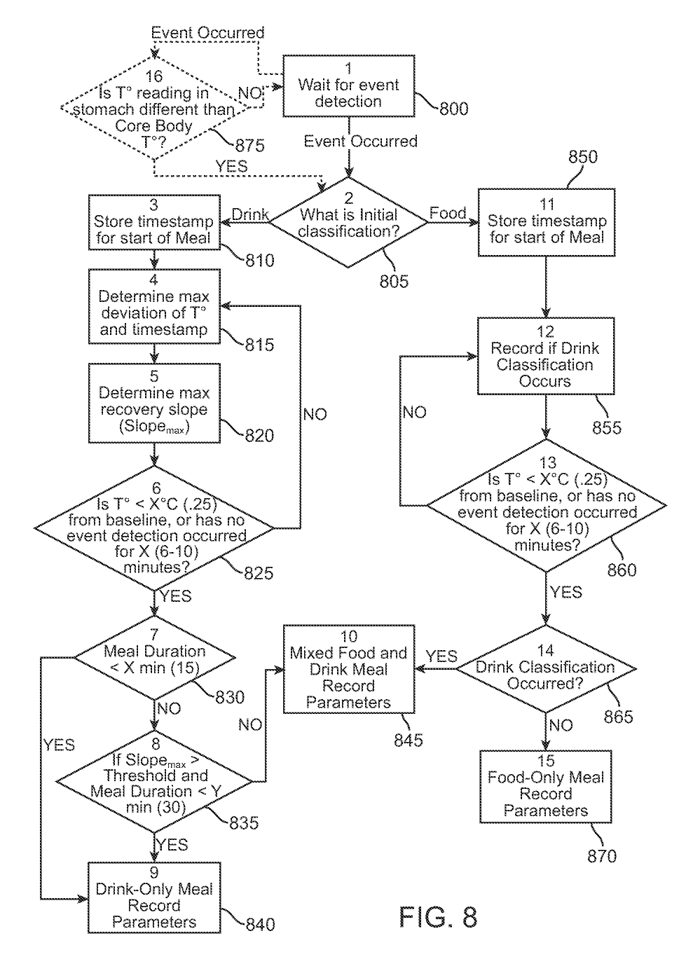

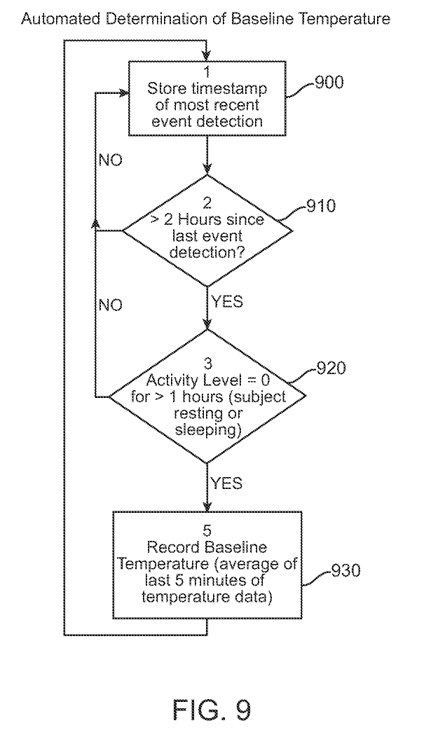

FIG. 8 illustrates an algorithm for defining a meal according to another embodiment of the present invention. Although this algorithm references the use of the temperature-based event classification algorithm described above, it may be implemented with any event classification algorithm that produces an ingestion event detection and a classification of the event as food or drink. This algorithm includes the use of a baseline body temperature and parameters related to the temperature deviation to provide a meal classification. A method of determining a baseline body temperature, such as is used here, is shown in FIG. 9.

FIG. 9 illustrates a method for automatically determining and updating a baseline body temperature, or core body temperature. This core body temperature measurement can be taken from a thermistor in the body of the implantable device, which is preferably attached to the inside wall of the stomach, or from a second thermistor that is not within the gastric lumen on the lead of the temperature sensor. Temperature measurements are taken periodically and stored temporarily for use in updating the baseline temperature value. Each time an event detection occurs, a time stamp is recorded that represents the time of the most recent event detection (step 900). Two criteria should be met in order to record or update the baseline temperature value: it should have been more than 2 hours since last event detection (step 910) and the activity level should be at a minimum for at least 1 hour (step 920). The minimum activity level corresponds to resting or sleeping by the patient. When these two criteria are met, then the baseline temperature value is updated using the average of the recently stored temperature measurements (step 930). In some embodiments, the average is taken from the most recent 5 minutes of temperature data.

Returning to the algorithm illustrated in FIG. 8, initially the controller waits for an event detection to occur (step 800). When an ingestion event is detected, it is classified (step 805). If the first event is classified as drink, the controller goes to step 810, otherwise to step 850, for a food classification. In both steps 810 and 850, a timestamp is stored for the start of a meal. In some embodiments, the controller may have access to a core body temperature measurement, which may be used prior to step 805 to verify the event detection by comparing the core body temperature measurement to the stomach temperature measurement in step 875. This comparison screens out event detections due to physiological changes that affect the core body temperature. For example, exercise, cyclical body temperature changes and other temperature changes unrelated to eating, such as fever due to illness, may all result in variations in the measured stomach temperature. However, classifying such temperature changes as ingestion events, and, particularly, providing therapy based on such classification (in those embodiments that incorporate therapy), would not be desirable.

Thus, in step 875, if the temperatures are in agreement within a set tolerance (i.e. ±x ° C., where x, for example, may be 0.25), then the event detection will be rejected and the controller will return to waiting mode (step 800). If the temperature difference is greater than the set tolerance, the event detection will be confirmed and classification in step 805 will proceed.

Next, following the steps for a drink event, the maximum deviation of the stomach temperature from the baseline and the timestamp for that maximum is determined (step 815). Following this point of maximum deviation, in step 820, the maximum slope as the signal returns to baseline is stored (Slopeₘₐₓ).

The meal is determined to be finished when the current temperature is within 0.25° C. from baseline, or when no event detection has occurred for a given number of minutes x (optimally 6 to 10 minutes) (step 825). In step 830, the meal duration is determined. If the duration is less than 15 minutes, then the meal is classified as drink only (step 840). If the meal duration is longer than 15 minutes, but less than 30 minutes, and the maximum recovery slope is greater than a threshold (step 835), there is also a classification of drink only (the purpose of this additional criteria is to detect large drinks). In some embodiments, the average recovery slope, the median recovery slope or the variance of the recovery slope may be used instead of the maximum recovery slope. If neither step 830, nor step 835 results in a drink only classification, the meal is classified as mixed food and drink (step 845).

As described above, when the first ingestion event is classified as food, a timestamp for the event is stored in step 850. The controller then records if a drink classification occurs in any subsequent ingestion events (step 855), while waiting for the criteria to be met for the end of the meal according to step 860, which is the same criteria as in step 825. When this criteria is met, the controller determines if a drink classification occurred (step 865). If there was a drink classification, then the meal is classified as mixed food and drink (step 845). If not, then the meal is classified as food only (step 870).

According to embodiments of the present invention, the classification of a meal and the determination of the end of a meal, as discussed above, may be used to control therapy. If the end of a meal cannot be precisely determined, it may be useful to employ a refractory period, during which the event classification algorithm may detect an event, but not trigger therapy. Refractory periods are particularly useful because the stomach temperature may not equilibrate to core body temperature for some time following the end of food ingestion, for example, in some cases it may take as long as 1.5 hours for the stomach to return to core body temperature. Therefore, it is advantageous to determine the end of ingestion based on other temperature signal characteristics, rather than relying entirely on a return to the baseline temperature. Signal characteristics that may be used include reduction in high frequency components of the temperature signal and variance of the temperature signal, among others.

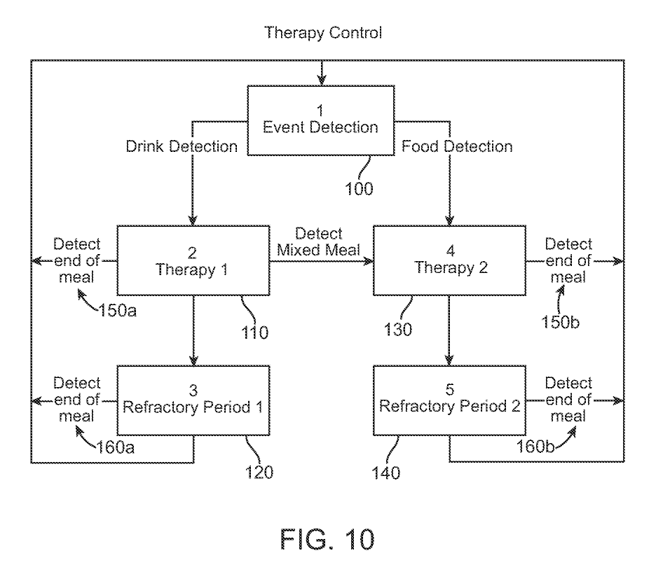

FIG. 10 illustrates one way that therapy may be tailored based on detection of the end of a meal, or meal classification, according to an embodiment of the present invention. Following event detection (step 100), a therapy is initiated. The type of therapy may depend on whether the event was classified as food or drink (steps 110 and 130). Following therapy there may be a nominal refractory period during which no therapy may be delivered. Such refractory periods may also be tailored to the event classification (steps 120 and 140) and the length of the refractory period may be programmed for the individual patient. If the end of a meal is detected prior to the end of therapy the processor may end or shorten the therapy, and skip the refractory period (paths 150a and 150b). If the end of a meal is detected prior to the end of the refractory period, the refractory period may be immediately ended (paths 160a and 160b). The therapy controller would then be ready to respond to another event detection. If the patient has been eating continuously through the therapy and refractory periods, the system may detect a new ingestion event and begin a new round of therapy. In an alternate embodiment, the system may disallow additional rounds of therapy until the meal that triggered the first round of therapy has ended.

In another embodiment of the present invention, the system allows up to 8 meal and/or therapy sessions to be defined by the user. These sessions allow the clinician to program periods of time during the day when a patient is likely to eat, and these periods can be individualized to the patient's schedule. Each session has a programmable eating therapy (responsive to the temperature sensor), drinking therapy (responsive to the temperature sensor), and time therapy (based on the clock). In addition, each therapy can be programmed off for any particular session. The timed therapy is typically a low level “conditioning” therapy which would condition the patient to start feeling full before a meal has started. The eating and drinking therapies will preempt the timed therapy when both are programmed on. The disallowed sessions (i.e., the time between each of the planned eating windows) will only have eating and drinking therapies; the timed therapy will be forced off. Sensor-based therapies will continue until completion when a new session starts, but time-based therapies would be cancelled for the session if a sensor-based therapy is already in progress.

The consumption classification algorithm could be used to trigger any therapy at the start or end of a meal. This therapy could involve other electrical stimulation that could lead to behavior modification, such as stimulation that would lead to discomfort, or gastrointestinal stimulation to treat diabetes. The consumption classification algorithm could also be used to trigger a patient warning, a physician notification, or useful diagnostics for the patient and physician.

The event and meal classification systems described above are based upon several parameters of the temperature data that is collected from the temperature sensor, accordingly embodiments of the present invention provide a method of preparing a classification system for patient ingestion in order to generate those parameters. Preparation of the system begins by providing training sets of temperature data to a classification algorithm. The training data sets consist of 32-sample sequences of temperature data that have been labeled with their corresponding activities (i.e., no consumption, eating, and drinking). In order to be effective, the classification system is trained using temperature waveforms that are representative of those that the final system will measure, meaning that the heat model and signal conditioning match. A broad data set, containing a variety of daily activities and foods representative of the target population for the implant, is preferred. The parameters to be generated are the event threshold, the seven feature weights used for the food and drink classification (described above), and the bias for this classification.

To establish the event threshold parameter, the mean temperatures for the samples 1 through 10 and 11 through 20 are calculated for each 32-sample waveform in the training set, and the absolute difference in the means is taken. The event threshold is calculated as 6 times the standard deviation of the screening function values from the no consumption classification. The resulting threshold is checked against the current data to find false positives and false negatives. False positives are highly undesirable, and cause to adjust the parameter selection criteria; while false negatives are more likely, but less problematic. In some embodiments, the data may be pre-processed to mimic the real data that will be encountered by the final system, such as by filtering, clipping, subsampling, and/or converting the data to a fixed-point format. As indicated above, pre-processing of the actual patient data collected during operation of the device may also be useful to remove noise or unwanted artifacts.

To establish the bias and feature weights, the features are calculated for all waveforms in the training set that are tagged as eating or drinking. Based upon these initial calculations, a set of features is calculated that will maximally separate the eating and drinking waveforms according to their features. In the current implementation, this is done using a support vector machine (SVM) library (e.g., with MATLAB®). The SVM calculated with a linear kernel describes a hyperplane that maximizes the distances between the feature vectors and the hyperplane. The classifier at this point can be described with: h(x)=sign(−b+Σᵢαᵢx′vᵢ). Here, x is a vector of features from a waveform being classified, vᵢ is each of the support vectors, and αᵢ is the associated coefficient. Because this is a linear kernel, the coefficient and support vectors can be pre-computed and reduced to a single set of weights: wᵢ=Σⱼαᵢvᵢⱼ. As described for the event threshold parameter, the data here may also be pre-processed to resemble real data more closely.

The embodiments presented above are examples of a learning method for classification. A learning method is beneficial when the signal being classified is very complex and the parameters that best differentiate the classifications are unknown. However, the accuracy of the classification algorithm is dependent upon the training data being representative of the total population of signals. Another advantage of such a learning method is that the classification algorithm can be tailored to the individual, if the support vector machine is trained using data from a single individual. This personalization would help take into account differences in eating habits, as well as gastric motility, which would provide greater accuracy in detection and classification, and improve overall treatment of the patient. Other alternate embodiments of the present invention include reducing the number of parameters that are part of the support vector machine calculation, based upon their effectiveness in separating the data. Also contemplated is the possibility of using greater or fewer parameters than the seven described above, as well as combining other classification strategies with the support vector machine approach.

In an alternate embodiment of the invention, the temperature sensor is placed at the entry of the stomach from the esophagus; this region is called the cardia. This placement allows more distinct sensing of each ingestion event, which is an advantage when multiple foods and drinks are swallowed within a short period of time. Each intake is accompanied by a temperature deviation that represents only that single ingestion event. When the sensor is positioned more centrally along the stomach wall, temperature deviations are a composite of multiple events, where each additional event produces less change as the bulk of substance in the stomach increases. Thus in this alternate embodiment, total consumption is proportional to the number of temperature deviations recorded and a meal is defined by the temperature deviations over time. For example, a first deviation would indicate the start of a meal, and the end of the meal would be determined by a period of time passing (e.g. x minutes) during which no temperature deviations occurred.

What is claimed is:

1. A method of classifying ingestion by a patient, the method comprising:
obtaining a plurality of stomach temperature sample values associated with a plurality of time intervals;
determining whether an ingestion event has occurred using the stored temperature values in order to determine whether classification is to be performed; and
classifying the ingestion event as eating or drinking using the stored temperature values.

2. The method of claim 1, further comprising storing the temperature values in a buffer, wherein the buffer stores a predetermined number of temperature values that define a sampling window.

3. The method of claim 2, wherein the step of determining whether an ingestion event has occurred comprises:
segmenting the sampling window into first, second and third time periods;
determining first and second averages of the temperature values for the first and second time periods;
comparing the first and second averages; and
determining whether the difference between the first and second averages exceeds a predetermined threshold.

4. The method of claim 2, wherein the step of classifying the ingestion event comprises analyzing features of the temperature values in the sampling window.

5. The method of claim 4, wherein the step of classifying the ingestion event further comprises using a linear separator to classify the ingestion event.

6. The method of claim 4, wherein the step of classifying the ingestion event further comprises using a non-linear separator to classify the ingestion event.

7. The method of claim 4, wherein the step of classifying the ingestion event further comprises weighting each of the analyzed features with an associated weight.

8. The method of claim 4, wherein the analyzed features include more than two of the following:
a mean of the temperature values;
a sum of the absolute values of sample-to-sample temperature differences;
a variance of the temperature values;
an area under a back half of a waveform defined by the temperature values in the sampling window;
an energy in a front half of the waveform;
an energy in the back half of the waveform; and
a maximum temperature difference of the temperature values.

9. The method of claim 1, wherein the step of determining whether an ingestion event has occurred and the step of classifying the ingestion event are performed using a single set of temperature values that define a single sampling window.

10. The method of claim 1, wherein the step of determining whether an ingestion event has occurred is performed using a first set of temperature values that define a first sampling window and the step of classifying the ingestion event is performed using a second set of temperature values that define a second sampling window.

11. The method of claim 1, further comprising obtaining additional temperature values and updating the buffer with the additional temperature values when it is determined that the temperature values are not to be classified or that an ingestion event has not occurred.

12. A method of classifying a meal ingested by a patient, the method comprising:
detecting a first ingestion event using at least one sensor disposed within a patient;
starting a meal timer in response to the event detection;
classifying the first ingestion event and recording the classification;
detecting and classifying subsequent ingestion events and recording the classifications until a predetermined period of time has passed without an event detection;
recording a meal duration in response to the time without an event detection; and
classifying the meal in response to signals from the at least one sensor.

13. The method of claim 12, wherein classifying the ingestion event comprises classifying the event as eating or drinking.

14. The method of claim 12, wherein classifying the meal comprises classifying the meal as food only, drink only or mixed food and drink.

15. The method of claim 12, further comprising determining an activity level of the patient, wherein the meal classification is set to drink only in response to the activity level of the patient indicating that the patient is exercising.

16. The method of claim 12, wherein the meal classification is set to drink only where classification of the first ingestion event is drink and the meal duration is shorter than a predetermined period.

17. A method of classifying a meal ingested by a patient, the method comprising:
obtaining a baseline stomach temperature of the patient;
waiting for an ingestion event;
detecting a first ingestion event; and
classifying the first ingestion event as food or drink and storing the classification;
where the classification of the first ingestion event is drink, determining and storing a maximum deviation of the stomach temperature from the baseline temperature, determining and storing a maximum recovery slope of the stomach temperature, determining an end of the meal and a meal duration, determining whether the recovery slope exceeds a predetermined threshold, and classifying the meal as drink only or mixed food and drink; and
where the classification of the first ingestion event is food, determining if a subsequent ingestion event is classified as drink, determining an end of the meal and classifying the meal as food only or mixed food and drink.

18. The method of claim 17, wherein determining the end of the meal comprises determining that the stomach temperature is within a predetermined range of the baseline temperature or that no event detection has occurred within a predetermined period of time.

19. The method of claim 17, further comprising storing a timestamp for a start of the meal.

20. The method of claim 17, wherein determining the end of the meal includes storing a timestamp of the end of the meal.

21. The method of claim 17, wherein the meal classification is set to drink only where the classification of the first ingestion event is drink and the meal duration is less than a first predetermined duration.

22. The method of claim 17, wherein the meal classification is set to drink only where the classification of the first ingestion event is drink, the meal duration is less than a second predetermined duration and the recovery slope exceeds a predetermined threshold.

23. The method of claim 17, further comprising obtaining a stomach temperature value when the first ingestion event is detected, comparing the temperature value to a core body temperature, and determining whether to accept the first ingestion event or to return to waiting for an ingestion event.

24. The method of claim 17, wherein obtaining the baseline stomach temperature of the patient comprises:
storing a timestamp of the most recent event detection;
determining if a predetermined period of time has passed since the most recent event detection;
determining an activity level of the patient; and
when the predetermined period of time has passed and the activity level of the patient is low, recording stomach temperature values over a period of time and averaging the temperature values to obtain a baseline stomach temperature.

25. A method of treatment of a patient, comprising:
detecting a first ingestion event;
classifying the ingestion event as food or drink;
where the ingestion event is classified as drink, providing a first therapy to the patient; and
where the ingestion event is classified as food, providing a second therapy to the patient.

26. The method of claim 25, further comprising providing a first refractory period to the patient after the first therapy and providing a second refractory period to the patient after the second therapy.

27. The method of claim 26, further comprising ending the first or second therapies or the first or second refractory periods when an end of a meal is detected.

28. The method of claim 25, further comprising:
detecting subsequent ingestion events, wherein the first and subsequent ingestion events define a meal;
classifying the meal; and
where the first ingestion event is classified as drink and the meal is classified as mixed food and drink, ending the first therapy to the patient and providing the second therapy to the patient.

29. A system for classifying ingestion by a patient comprising:
a temperature sensor adapted to be placed in the stomach of the patient;
a storage medium connected to the sensor for storing temperature values; and
a processor connected to the storage medium that is configured to analyze the temperature values, wherein the processor includes a module for determining whether the temperature values are to be classified, a module for determining whether an ingestion event has occurred and a module for classifying the ingestion event as eating or drinking.

30. The system of claim 29, wherein the processor includes a tangible medium embodying instructions for analyzing the temperature values, determining whether the temperature values are to be classified, determining whether an ingestion event has occurred and classifying the ingestion event.

31. A system for classifying a meal ingested by a patient comprising:
a temperature sensor adapted to be placed in the stomach of the patient;
a meal timer;
an activity sensor;
a storage medium connected to the temperature sensor, the meal timer and the activity sensor; and
a processor connected to the storage medium that is configured to analyze temperature values, timestamps and activity level data stored in the storage medium to classify the meal.

32. A system for classifying a meal ingested by a patient comprising:
a temperature sensor adapted to be positioned in the stomach of the patient;
a storage medium connected to the temperature sensor; and
a processor connected to the storage medium that is configured to analyze temperature values stored in the storage medium to classify the meal, wherein the processor includes:
a first module for determining a baseline stomach temperature of the patient,
a second module for classifying a first ingestion event as food or drink based on the temperature values, and
a third module for classifying the meal, where when the classification of the first ingestion event is drink, the third module determines and stores a maximum deviation of the stomach temperature from the baseline temperature, determines and stores a maximum recovery slope of the stomach temperature, determines an end of the meal and a meal duration, determines whether the recovery slope exceeds a predetermined threshold, and classifies the meal as drink only or mixed food and drink, and when the classification of the first ingestion event is food, the third module determines if a subsequent ingestion event is classified as drink, determines an end of the meal and classifies the meal as food only or mixed food and drink.

33. A system for treatment of a patient comprising:
a temperature sensor adapted to be positioned in the stomach of the patient;
a storage medium coupled to the temperature sensor;
a therapeutic device adapted to provide at least one therapy to the patient; and
a processor coupled to the storage medium and the therapeutic device that is configured to analyze temperature values stored in the storage medium to classify the meal and to control the therapeutic device based on the classification.

34. A system for classifying ingestion by a patient comprising:
means for obtaining a plurality of stomach temperature sample values;
means for storing the temperature values; and
means for analyzing the stored temperature values, wherein the means for analyzing includes means for determining whether the stored temperature values are to be classified, means for determining whether an ingestion event has occurred using the stored temperature values, and means for classifying the ingestion event as eating or drinking using the stored temperature values.

35. A method of preparing a classification system for patient ingestion comprising:
providing training sets of data to a classification algorithm, wherein the training sets correspond to known activities;
determining a set of features of the temperature data;
determining a set of weights corresponding to the set of features using the data and the corresponding known activities; and
deriving a classification algorithm from the set of features and the set of weights.

36. The method of claim 35, wherein the data comprises temperature data and further comprising determining an event parameter threshold and a bias value and incorporating the event parameter threshold and the bias value into the classification algorithm.

37. The method of claim 36, wherein determining the bias value and determining the set of weights includes using a support vector machine.

38. The method of claim 36, wherein determining the bias value and determining the set of weights includes optimizing the bias value and the set of weights to provide a maximum separation between the waveforms corresponding to eating and drinking.

39. The method of claim 35, wherein the known activities include no consumption, eating, and drinking, wherein eating and drinking are defined as screening functions.

40. The method of claim 39, wherein the training sets comprise 32-sample data sets.

41. The method of claim 39, wherein the data comprises temperature data and wherein determining the event threshold parameter includes calculating the mean temperatures for first and second sample subsets of the data sets corresponding to each of the screening functions, determining the absolute difference in the mean temperatures, determining the standard deviation of the screening function values from the no consumption values, and determining the event threshold.

42. The method of claim 39, wherein the set of features to which the weights correspond include more than two of the following:
a mean of the temperature values;
a sum of the absolute values of the sample-to-sample temperature differences;
a variance;
an area under a back half of a waveform defined by the temperature values in the sampling window;
an energy in a front half of the waveform;
an energy in the back half of the waveform; and
a maximum temperature difference.

43. A method of providing therapy to a patient comprising:
providing a therapy device with a schedule of allowed and disallowed periods for the patient;
for each allowed period according to the schedule, applying a first therapy to the patient at the start of the allowed period, detecting an ingestion event with at least one temperature sensor disposed within the patient, and classifying the ingestion event as food or drink;
where the ingestion event during the allowed period is classified as drink, stopping the first therapy and providing a second therapy to the patient;
where the ingestion event during the allowed period is classified as food, stopping the first therapy and providing a third therapy to the patient; and
for each disallowed period, detecting an ingestion event with the at least one temperature sensor and classifying the ingestion event as food or drink;
where the ingestion event during the disallowed period is classified as drink, providing the second therapy to the patient;
where the ingestion event during the disallowed period is classified as food, providing the third therapy to the patient.
